(19) United States
(12) Patent Application Publication
(10) Pub. No.: US 2011/0015413 A1
(43) Pub. Date: Jan. 20, 2011

(54) PROCESS FOR PREPARING CRYSTALLINE CALCIUM MUPIROCIN DIHYDRATE

(76) Inventors: Nafis Defterdarovic, Zagreb (HR); Marijan Tudja, Zagreb (HR); Drazen Cavuzic, Zagreb (HR)

Correspondence Address: KENYON & KENYON LLP, ONE BROADWAY, NEW YORK, NY, 10004, US

(21) Appl. No.: 12/863,657

(22) PCT Filed: Dec. 30, 2008
(86) PCT No.: PCT/GB2008/004307

§ 371 Date: Sep. 7, 2010

Related U.S. Application Data
(60) Provisional application No. 61/023,350, filed on Jan. 24, 2008.

(51) Int. Cl. C07D 407/06

(52) U.S. Cl. 549/414

(57) ABSTRACT

Provided herein are methods of preparing crystalline mupirocin calcium dihydrate comprising combining solid mupirocin, solid calcium carbonate, and water in an amount sufficient to form a heterogeneous mixture and precipitate crystalline mupirocin calcium dihydrate from the mixture. In some embodiments, the methods produce nanocrystalline mupirocin calcium dihydrate, suitable for use in pharmaceutical formulations.

### FIELD OF THE INVENTION

The present invention relates generally to crystalline mupirocin calcium dihydrate and processes for its production. In particular the invention relates to nanocrystalline mupirocin calcium dihydrate and the use of heterogeneous reaction mixtures for its production.

### BACKGROUND

The following description is provided to assist the understanding of the reader. None of the information provided or references cited is admitted to be prior art to the present invention.

Mupirocin, also known as pseudomonic acid A, is an antibiotic produced by cultures of Pseudomonas fluorescens under aerobic conditions. Mupirocin has the structure A below.

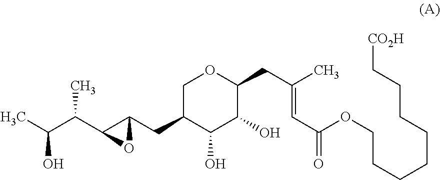

Mupirocin is a somewhat unstable compound, which, under acidic or alkaline conditions, undergoes rearrangement to the bicyclic rearrangement products I and II, below.

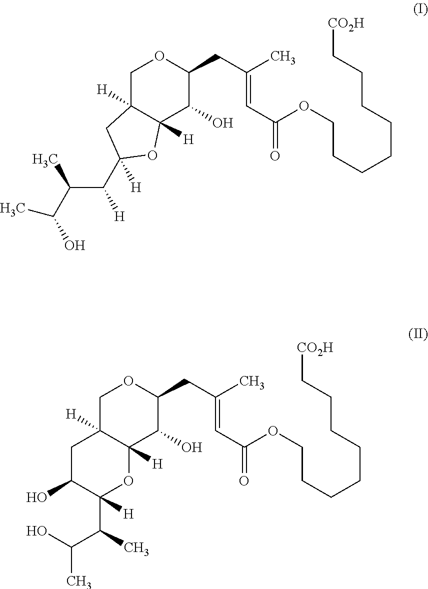

Such rearrangements may result from an autocatalytic reaction involving the mupirocin carboxylic acid group. The fragile nature of mupirocin has largely limited pharmaceutical formulations of the drug to ointments for topical use of skin infections and the like. Much research has focused on discovering more stable forms of mupirocin.

Due to the instability of mupirocin, finding a form of this antibiotic that could be formulated at a sufficiently high purity for pharmaceutical use was challenging. As disclosed during prosecution of U.S. Ser. No. 07/072,683, the parent of U.S. Pat. No. 4,916,155 and grandparent of U.S. Pat. No. 5,191,093, at least 23 inorganic and organic salts were prepared or attempted to be prepared in order to find one useful crystalline form of mupirocin for pharmaceutical production (See Declaration of Norman Harold Rogers, paragraphs 11 and 12.) Six of the 23 salts could not be isolated at all, and most of the salts which could be formed were isolated as oils or amorphous powders. None of the non-crystalline salts possessed adequate stability for pharmaceutical use. The two salts which were crystalline (lithium and S-benzylthiouronium) also turned out to be unsuitable for pharmaceutical use.

Calcium mupirocin was investigated as one of the 23 original salts, but the original experiments failed to produce any crystalline material. Initial attempts to prepare a calcium salt of mupirocin “gave a hygroscopic, sticky, impure solid” (Id., paragraphs 13 and 16(i).) Further attempts in which mupirocin free acid was dissolved in water along with calcium carbonate or calcium hydroxide gave only non-crystalline powders of moderate purity (84% and 89-90%, respectively). (Id., paragraph 16(11) and (iii).) Attempts to crystallize these samples of calcium mupirocin from water and a variety of organic solvents were unsuccessful. (Id., paragraph 17.) The amorphous calcium salt of mupirocin has been described as “sparingly water soluble material having a low melting point and poor thermal stability” and unsuitable for pharmaceutical use. Eventually, crystalline mupirocin calcium dihydrate was formed by chance during stability studies of the free acid form of the antibiotic. (Id., paragraph 22.)

U.S. Pat. No. 5,191,093 discloses formation of crystalline mupirocin calcium dihydrate by one of two general methods. In a first method, the free acid form of mupirocin in a 50% aqueous solution of methanol is neutralized with calcium oxide followed by removal of the organic solvent and crystallization from water (Examples 1-3). In a second method, a salt of mupirocin such as the lithium, sodium or potassium salt is exchanged for calcium in an aqueous solution (Examples 4-6). Thus, both methods utilize homogenous systems in which the mupirocin is solubilized for reaction either by use of an organic solvent or formation of a salt of mupirocin having greater water solubility than the calcium salt. Neither U.S. Pat. No. 5,191,093 nor the Declaration of Rogers from the prosecution of U.S. Ser. No. 07/072,683 disclose any methods for directly producing crystalline mupirocin calcium dihydrate from a heterogeneous system.

WO 2003/065975 describes a process for preparing crystalline mupirocin calcium dihydrate which includes preparing a solution of pseudomonic acid A in a water-immiscible solvent, and combining the solution with a solution or a suspension of a calcium C₂ to C₁₂ organic carboxylate in an aqueous solvent, to form an aqueous and a non-aqueous phase, wherein mupirocin calcium dihydrate precipitates from the aqueous phase. 2-Ethyl-hexanoate is disclosed as a preferred calcium carboxylate.

### SUMMARY

Processes are described herein for preparing crystalline mupirocin calcium dihydrate in a heterogeneous reaction system. The processes are operationally simple and economical, requiring only mupirocin free acid, calcium carbonate and water. No organic solvents are required, and the primary byproduct, carbon dioxide is readily removed. Furthermore, processes of the invention provide nanocrystalline mupirocin calcium dihydrate having a relatively uniform particle size. Thus, the mild reaction conditions lead to mupirocin calcium dihydrate in good yield and high purity, suitable for use in pharmaceutical formulations.

### BRIEF DESCRIPTION OF THE DRAWINGS

FIG. 1: Thermal field scanning electron microscopy picture of calcium mupirocin dihydrate (10000× magnification).

FIG. 2: FE SEM image of calcium mupirocin dihydrate (550× magnification).

### DETAILED DESCRIPTION

Methods of preparing crystalline mupirocin calcium dihydrate have been found which, surprisingly, do not require the use of organic solvents or calcium ion exchange of water-soluble salt forms of mupirocin. Instead, the present methods utilize a unique steady state reaction system to control formation of the mupirocin calcium salt and directly crystallize the mupirocin calcium dihydrate from the aqueous reaction medium. In particular, a heterogeneous mixture is formed by combining solid mupirocin, solid calcium carbonate, and water. The amount of the water is controlled to avoid a homogenous solution and maintain heterogeneity during formation of the crystalline mupirocin calcium dihydrate.

In the description herein, a number of terms are utilized extensively. Definitions are provided to facilitate understanding of the invention. The terms defined below are more fully defined by reference to the specification as a whole.

Solid mupirocin is a solid form of the free acid of mupirocin.

Crystalline mupirocin calcium dihydrate is the calcium dihydrate salt form of mupirocin containing about 5%, preferably 4%, 3%, 2% or 1%, or less by weight of non-crystalline calcium mupirocin.

Nanocrystalline mupirocin calcium dihydrate is a form of mupirocin calcium dihydrate having a mean particle size of less than 1 μm.

We present as a feature of the invention a method of manufacturing crystalline mupirocin calcium dihydrate the method comprising combining solid mupirocin, solid calcium carbonate, and an amount of water sufficient to form a heterogeneous mixture and the amount of water also being sufficient to precipitate the crystalline mupirocin calcium dihydrate formed from the mixture.

In some embodiments, the solid mupirocin is added to a mixture of calcium carbonate and water. The mupirocin is used as the free acid and may be amorphous or crystalline. In the present methods, the molar amount of solid calcium carbonate may vary somewhat in relation to the molar amount of solid mupirocin, but will generally be about 1:1. The molar ratio of mupirocin to calcium carbonate can, for example, range from about 0.95 to about 1.05. Typically, the molar amount of calcium carbonate to mupirocin ranges from about 1 to 1.03.

The amount of water in the present methods must be enough to provide for good mixing of the reaction, but small enough to maintain a heterogeneous mixture throughout the reaction. For example, the amount of water can range from about 6 mL/g of mupirocin to about 20 ml/g of mupirocin, and preferably ranges from about 10 mL/g of mupirocin to about 18 ml/g of mupirocin. While not wishing to be bound by theory, it is believed that these amounts of water allow a steady state reaction to occur where only a portion of the mupirocin and calcium carbonate are in solution and available to form the mupirocin calcium salt. The amount of water used in the reaction is also small enough that the mupirocin calcium salt, once formed, freely precipitates from solution as the dihydrate, protecting it from further rearrangement reactions.

While cosolvents may be used in the present methods, they are not required and must be used in amounts so as to maintain the heterogeneous mixture throughout the reaction. Suitable cosolvents include, e.g., C₁₋₄ alcohols, ketones, and water miscible ethers such as, but not limited to, methanol, ethanol, propanol, butanol, acetone, and dioxane. In some embodiments, the methods are performed in water in the absence of cosolvents.

The temperature at which the present methods are carried out may vary. Typically, the heterogeneous mixture is held at a temperature ranging from about 5° C. to about 35° C. during the reaction (i.e., during the precipitation of the crystalline mupirocin calcium dihydrate). In some embodiments, the reaction temperature ranges from about 10° C. to about 35° C., from about 10° C. to about 30° C., from about 15° C. to about 30° C. While reaction temperatures from about 20° C. to about 25° C. are preferred, a variety of other temperatures are suitable. It is within the skill in the art to select an appropriate temperature for the reaction in view of the guidance provided herein.

In some embodiments of the present methods, the mupirocin calcium dihydrate produced by the methods described herein is nanocrystalline mupirocin calcium dihydrate. Typically, the nanocrystalline mupirocin calcium dihydrate has a mean particle size of about 100 nm to about 900 nm. In some embodiments, the nanocrystalline mupirocin calcium dihydrate has a mean particle size of about 100 nm to about 700 nm. In some embodiments, the nanocrystalline mupirocin calcium dihydrate fuse together and are present as agglomerates having a mean particle size of from about 5 μm to about 15 μm.

The present methods of preparing crystalline mupirocin calcium dihydrate include isolating and drying the precipitated crystalline mupirocin calcium dihydrate. In some embodiments, the precipitated nanocrystalline mupirocin calcium dihydrate is isolated (e.g., by filtration) and dried. During drying the mupirocin calcium dihydrate may form aggregates. The aggregates may have a mean particle size of from about 30 μm to about 400 μm. The crystalline mupirocin dihydrate is typically dried so as to maintain the dihydrate crystalline form. However, it may also be dried under more vigorous conditions to provide the anhydrous form.

The present methods provide precipitated crystalline mupirocin calcium dihydrate in high purity and may be at least 90% pure by weight. In some embodiments the precipitated crystalline mupirocin calcium dihydrate is at least 95%, at least 96%, or at least 97% pure by weight. Thus, the precipitated crystalline mupirocin calcium dihydrate may contain less that 2 wt % each of Pseudomonic acid B, Pseudomonic acid C, Pseudomonic acid D, rearrangement product I, rearrangement product II, or calcium salts thereof.

Aspects and embodiments of the invention described herein are illustrated by the following non-limiting examples.

### EXAMPLES

### Example 1

Mupirocin in the form of the free acid was isolated from Pseudomonas fluorescens Biotype A, essentially according to known procedures. Calcium mupirocin dihydrate was synthesized from solid mupirocin (free acid) as follows.

### Example 1.1

Into a glass flask equipped with agitator were charged deionised water (70 mL), calcium carbonate (0.4 g) and mupirocin (4.0 g) and stirred for 70 hours at room temperature. Mupirocin calcium dihydrate was filtered off using a Büchner funnel, washed with 3×10 mL deionised water and dried at atmospheric conditions to the constant weight. Yield 3.7 g (melting point 137° C.)

### Example 1.2

Into a glass flask equipped with agitator were charged deionised water (113 mL), acetone (10 mL) calcium carbonate (1.0 g) and mupirocin (10.1 g) and stirred for 73.5 hours at room temperature. Mupirocin calcium dihydrate was filtered off using a Büchner funnel, washed with 3×10 mL deionised water and dried at atmospheric conditions to the constant weight. Yield 9.7 g.

### Example 1.3

Into a glass flask equipped with agitator were charged deionised water (22.5 mL), methanol (2.0 mL), calcium carbonate (0.2 g) and mupirocin (2.0 g) and stirred for 25 hours at room temperature. Mupirocin calcium dihydrate was filtered off using a Büchner funnel, washed with 2×3 mL deionised water and dried at atmospheric conditions to the constant weight. Yield 1.9 g (melting point 136° C.).

### Example 1.4

Into a glass flask equipped with agitator were charged deionised water (22.5 mL), ethanol (2.2 mL), calcium carbonate (0.2 g) and mupirocin (2.0 g) and stirred for 25 hours at room temperature. Mupirocin calcium dihydrate was filtered off using a Büchner funnel, washed with 2×3 mL deionised water and dried at atmospheric conditions to the constant weight. Yield 1.8 g (melting point 136° C.).

### Example 1.5

Into a glass flask equipped with agitator were charged deionised water (22.5 mL), 1-propanol (2.0 mL), calcium carbonate (0.2 g) and mupirocin (2.0 g) and stirred for 25 hours at room temperature. Mupirocin calcium dihydrate was filtered off using a Büchner funnel, washed with 2×3 mL deionised water and dried at atmospheric conditions to the constant weight. Yield 1.7 g (melting point 137° C.).

### Example 1.6

Into a glass flask equipped with agitator were charged deionised water (22.5 mL), 2-propanol (2.0 mL), calcium carbonate (0.2 g) and mupirocin (2.0 g) and stirred for 24 hours at room temperature. Mupirocin calcium dihydrate was filtered off using a Büchner funnel, washed with 2×3 mL deionised water and dried at atmospheric conditions to the constant weight. Yield 1.9 g (melting point 136° C.).

### Example 1.7

Into a glass flask equipped with agitator were charged deionised water (22.5 mL), 2-butanol (2.0 mL), calcium carbonate (0.2 g) and mupirocin (2.0 g) and stirred for 24 hours at room temperature. Mupirocin calcium dihydrate was filtered off using a Büchner funnel, washed with 2×3 mL deionised water and dried at atmospheric conditions to the constant weight. Yield 0.4 g (melting point 135° C.).

### Example 1.8

Into a glass flask equipped with agitator were charged deionised water (16.5 mL), dioxane (1.0 mL), calcium carbonate (0.2 g) and mupirocin (2.0 g) and stirred for 24 hours at room temperature. Mupirocin calcium dihydrate was filtered off using a Büchner funnel, washed with 2×3 mL deionised water and dried at atmospheric conditions to the constant weight. Yield 1.8 g (melting point 136° C.).

### Example 1.9

Into a glass flask equipped with agitator were charged deionised water (87.5 mL), calcium carbonate (0.5 g) and mupirocin (5.0 g) and stirred for 74 hours at 6±1° C. Product was filtered off using a Büchner funnel, washed with 2×10 mL deionised water and dried at atmospheric conditions to the constant weight. Yield 4.4 g (melting point 78° C.).

### Example 1.10

Into a glass flask equipped with agitator were charged deionised water (88 mL), calcium carbonate (0.5 g) and mupirocin (5.0 g) and stirred for 72 hours at 15-16° C. Mupirocin calcium dihydrate was filtered off using a Büchner funnel, washed with 2×10 mL deionised water and dried at atmospheric conditions to the constant weight. Yield 4.4 g (melting point 133° C.).

### Example 1.11

Into a glass flask equipped with agitator were charged deionised water (88 mL), calcium carbonate (0.5 g) and mupirocin (5.0 g) and stirred for 72 hours at 18.5-19° C. Mupirocin calcium dihydrate was filtered off using a Büchner funnel, washed with 2×10 mL deionised water and dried at atmospheric conditions to the constant weight. Yield 4.6 g (melting point 132° C.).

### Example 1.12

Into a glass flask equipped with agitator were charged deionised water (88 mL), calcium carbonate (0.5 g) and mupirocin (5.0 g) and stirred for 72 hours at 23° C. Mupirocin calcium dihydrate was filtered off using a Büchner funnel, washed with 2×10 mL deionised water and dried at atmospheric conditions to the constant weight. Yield 4.6 g (melting point 136° C.).

### Example 1.13

Into a glass flask equipped with agitator were charged deionised water (88 mL), calcium carbonate (0.5 g) and mupirocin (5.0 g) and stirred for 72 hours at 24° C. Mupirocin calcium dihydrate was filtered off using a Büchner funnel, washed with 2×10 mL deionised water and dried at atmospheric conditions to the constant weight. Yield 4.7 g (melting point 135° C.).

### Example 1.14

Into a glass flask equipped with agitator were charged deionised water (88 mL), calcium carbonate (0.5 g) and mupirocin (5.0 g) and stirred for 72 hours at 25° C. Mupirocin calcium dihydrate was filtered off using a Büchner funnel, washed with 2×10 mL deionised water and dried at atmospheric conditions to the constant weight. Yield 4.8 g (melting point 134° C.).

### Example 1.15

Into a glass flask equipped with agitator were charged deionised water (88 mL), calcium carbonate (0.5 g) and mupirocin (5.0 g) and stirred for 72 hours at 27° C. Mupirocin calcium dihydrate was filtered off using a Büchner funnel, washed with 2×10 mL deionised water and dried at atmospheric conditions to the constant weight. Yield 4.5 g (melting point 127° C.).

### Example 1.16

Into a glass flask equipped with agitator were charged deionised water (87.5 mL), calcium carbonate (0.5 g) and mupirocin (5.0 g) and stirred for 72 hours at 30° C. Mupirocin calcium dihydrate was filtered off using a Büchner funnel, washed with 2×10 mL deionised water and dried at atmospheric conditions to the constant weight. Yield 4.6 g (melting point 132° C.).

### Example 1.17

Into a glass flask equipped with agitator were charged deionised water (87.5 mL), calcium carbonate (0.5 g) and mupirocin (5.0 g) and stirred for 9.5 hours at room temperature. Mupirocin calcium dihydrate was filtered off using a Büchner funnel, washed with 2×10 mL deionised water and dried at atmospheric conditions to the constant weight. Yield 3.9 g (melting point 130° C.).

### Example 1.18

Into a glass flask equipped with agitator were charged deionised water (87.5 mL), calcium carbonate (0.5 g) and mupirocin (5.0 g) and stirred for 9.5 hours under vacuum (0.1 bar absolute pressure) at room temperature. Mupirocin calcium dihydrate was filtered off using a Büchner funnel, washed with 2×10 mL deionised water and dried at atmospheric conditions to the constant weight. Yield 4.3 g (melting point 130° C.).

### Example 1.19

Into a 500 L glass lined reactor equipped with agitator and thermostat were charged deionised water (171 L) and calcium carbonate (1.24 kg). Temperature of the reactor content was adjusted at 23±1° C. and at that temperature mupirocin (12.0 kg) was added. Reaction mixture was stirred for 50 hours at the temperature 23±1° C. under vacuum (0.2-0.8 bar absolute). Mupirocin calcium dihydrate was isolated using a centrifuge, washed with 45 L of deionised water and dried in a fluid bed drier. Yield 11.6 kg of mupirocin calcium dihydrate.

A typical analysis of the product produced by the above process on an industrial scale is shown in Table 1. The composition of the product is notable for the extremely low amounts rearrangement products I and II and the high amount of calcium mupirocin dihydrate.

TABLE 1

| Component | Weight % |
|---|---|
| Mupirocin calcium dihydrate | 97.6 |
| Rearrangement product I of the | 0.146 |
| Pseudomonic acid A | |
| Rearrangement product II of the | 0.137 |
| Pseudomonic acid A | |
| Pseudomonic acid B | 0.00 |
| Pseudomonic acid C | 0.00 |
| Pseudomonic acid D | 1.34 |
| Total impurities | 2.03 |

### Example 2

The morphology of crystalline mupirocin calcium dihydrate produced according to the procedure of Example 2 was examined by thermal field scanning electron microscopy (FE SEM). An FE SEM microscope, model JSM-7000F, manufactured by Jeol Ltd., Japan, was used in the inspection of samples. The FE SEM microscope was coupled with an EDS/INCA 350 (energy dispersive X-ray analyzer) manufactured by Oxford Instruments Ltd. The samples inspected by FE SEM were not coated with a conductive layer.

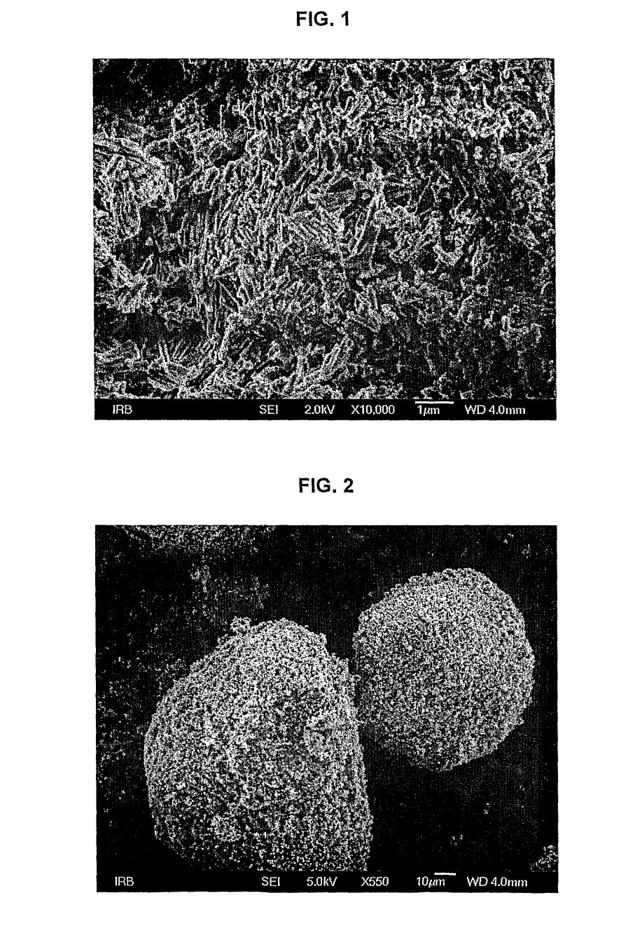

FIG. 1 shows the morphology of calcium mupirocin dihydrate crystals. Calcium mupirocin dihydrate crystallize in a columnar particle shape having a mean particle size from about 100 to about 700 nm. During the crystallisation process primary particles, i.e., crystals, are fused to form agglomerates (FIG. 2) having a mean particle size of about 10 μm. During the drying process, the agglomerates adhere to each other, forming aggregates having a mean particle size from about 30 μm to about 400 μm.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 atoms refers to groups having 1, 2, or 3 atoms. Similarly, a group having 1-5 atoms refers to groups having 1, 2, 3, 4, or 5 atoms, and so forth.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

While certain embodiments have been illustrated and described, it should be understood that changes and modifications can be made therein in accordance with ordinary skill in the art without departing from the invention in its broader aspects as defined in the following claims.

The average particle size can also be referred to as the D[4,3] value and is measured by means of low angle light scattering (LALLS) techniques, preferably using laser diffraction such as in a Malvern Mastersizer machine.

1. A method of manufacturing crystalline mupirocin calcium dihydrate the method comprising combining solid mupirocin, solid calcium carbonate, and an amount of water sufficient to form a heterogeneous mixture and the amount of water also being sufficient to precipitate the crystalline mupirocin calcium dihydrate formed from the mixture.

2. The method of claim 1 wherein the amount of water ranges from about 6 mL/g of mupirocin to about 20 ml/g of mupirocin.

3. The method of claim 1 wherein the amount of water ranges from about 10 mL/g of mupirocin to about 18 ml/g of mupirocin.

4. The method of claim 1 wherein the molar ratio of solid mupirocin to calcium carbonate ranges from about 0.95 to about 1.05.

5. The method of claim 4 wherein the molar ratio of solid mupirocin to calcium carbonate is about 1:1.

6. The method of claim 1 wherein the heterogeneous mixture is held at a temperature ranging from about 5° C. to about 35° C. during the reaction.

7. The method of claim 1 wherein the heterogeneous mixture is held at a temperature from about 20° C. to about 25° C. during the reaction.

8. The method of claim 1 wherein the method is carried out under reduced pressure.

9. The method of claim 8 wherein the pressure ranges from about 50 millibars to about 800 millibars.

10. The method of claim 1 wherein the crystalline mupirocin calcium dihydrate is nanocrystalline mupirocin calcium dihydrate.

11. The method of claim 10 wherein the nanocrystalline mupirocin calcium dihydrate has a mean particle size of about 100 nm to about 900 nm.

12. The method of claim 10 wherein the nanocrystalline mupirocin calcium dihydrate has a mean particle size of about 100 nm to about 700 nm.

13. The method of claim 10 wherein the nanocrystalline mupirocin calcium dihydrate is present as agglomerates having a mean particle size of from about 5 μm to about 15 μm.

14. The method of claim 1 further comprising isolating and drying the precipitated crystalline mupirocin calcium dihydrate.

15. The method of claim 10 further comprising isolating and drying the precipitated nanocrystalline mupirocin calcium dihydrate.

16. The method of claim 15 wherein during drying the nanocrystalline mupirocin calcium dihydrate forms aggregates.

17. The method of claim 16 wherein the aggregates have a mean particle size of from about 30 μm to about 400 μm.

18. The method of claim 1 wherein the precipitated crystalline mupirocin calcium dihydrate is at least 90% pure by weight.

19. The method of claim 1 wherein the precipitated crystalline mupirocin calcium dihydrate is at least 95% pure by weight.

20. The method of claim 1 wherein the precipitated crystalline mupirocin calcium dihydrate contains less that 2 wt % each of Pseudomonic acid B, Pseudomonic acid C, Pseudomonic acid D, rearrangement product I, rearrangement product II, or calcium salts thereof.

21. The method of claim 15 further comprising drying the crystalline mupirocin calcium dihydrate to an anhydrous crystalline form.
